(19) United States
(12) Patent Application Publication
(10) Pub. No.: US 2026/0063595 A1
(43) Pub. Date: Mar. 5, 2026

(54) METHOD FOR CLASSIFYING RESINS

(71) Applicant: Toyo Tire Corporation, Itami-shi (JP)

(72) Inventor: Yohsuke Kobiki, Itami-shi (JP)

(73) Assignee: Toyo Tire Corporation, Itami-shi (JP)

(21) Appl. No.: 19/299,507

(22) Filed: Aug. 14, 2025

(30) Foreign Application Priority Data
Sep. 5, 2024 (JP) 2024-152976

(51) Int. Cl. G01N 30/12; B01D 53/02; G01N 30/02; G01N 30/72; G01N 30/86; G01N 33/44

(52) U.S. Cl. CPC G01N 30/12; B01D 53/025; G01N 30/7206; G01N 30/8624; G01N 33/442; B01D 2259/402; B01D 2259/414; G01N 2030/025; G01N 2030/125

(57) ABSTRACT

A method for classifying at least two or more resins, the method comprising: a purification step of removing volatile components by subjecting resins to be classified to a heat treatment at a temperature lower than each decomposition temperature; a decomposition step of subjecting the resins to be classified to a pyrolysis treatment at a temperature higher than each decomposition temperature to obtain pyrolysates of the resins; an analysis step of separating and analyzing the pyrolysates using a two-dimensional gas chromatograph mass spectrometer; a table creation step of creating a table by performing assignment of peaks appearing in each two-dimensional chromatogram obtained after the analysis step and alignment between the two-dimensional chromatograms; and a classification step of classifying resins by performing multivariate analysis based on the created table.

### BACKGROUND OF THE INVENTION

### Field of the Invention

The present invention relates to a method for classifying resins.

### Description of the Related Art

For example, many rubber products such as pneumatic tires are produced by vulcanizing a rubber composition as a raw material. In the rubber composition, many blending agents are added for improving physical properties, and many hydrocarbon-based resins are also blended.

Since resins have various applications in addition to blending agents for tires as materials for improving braking performance on a wet road surface (hereinafter, also referred to as “WETμ”), resins having various chemical structures have been developed. However, with regard to the chemical structure of a resin, it has been common to obtain rough information on monomer species by analysis by pyrolysis gas chromatography, and there has been a limit to the detailed structural classification. There is a possibility that the physical properties of a resin are greatly different depending on a slight difference in the type and amount of monomers, and there is a demand for a method in which detailed classification information is obtained and associated with the physical properties of a finally obtained vulcanized rubber.

Patent Document 1 mentioned below discloses a method for individually determining contents of respective resin components in a paste containing a plurality of resin components and an inorganic powder, the method including: a dissolution step in which the paste and an organic solvent are mixed to dissolve the resin components; a separation step in which the inorganic powder is separated from a solution obtained in the dissolution step to form a resin solution containing the resin components; a measurement step in which the resin solution is measured by pyrolysis gas chromatography to determine peak area values of pyrolysis products intrinsic to the respective resin components generated by pyrolysis of the respective resin components; a quantitative determination step in which contents of the respective resin components in the paste are calculated from the peak area values determined in the measurement step on the basis of a calibration curve representing a correlation between the contents and the peak area values of the intrinsic pyrolysis products; and between the separation step and the measurement step, a drying step in which the resin solution is dried by heating at a temperature equal to or higher than a boiling point of the organic solvent and equal to or lower than pyrolysis temperatures of the resin components to vaporize the organic solvent and form a dried product containing the resin components, wherein in the measurement step, the dried product is measured by pyrolysis gas chromatography.

### PRIOR ART DOCUMENT

### Patent Document

Patent Document 1: JP-B2-6950494

### SUMMARY OF THE INVENTION

As a result of intensive studies by the present inventors, it cannot be said that the above-described conventional technique is merely intended to quantify resin components and is not a detailed structural classification. Therefore, it is actually required to obtain detailed classification information of the resins and establish a method associated with the physical properties of the finally obtained vulcanized rubber.

The present invention has been made in view of the above circumstances, and an object thereof is to provide a resin classification method capable of classifying resins having various chemical structures.

The above object can be achieved by the following configurations. That is, the present invention relates to a method for classifying at least two or more resins (1), the method including: a purification step of removing volatile components by subjecting resins to be classified to a heat treatment at a temperature lower than each decomposition temperature; a decomposition step of subjecting the resins to be classified to a pyrolysis treatment at a temperature higher than each decomposition temperature to obtain pyrolysates of the resins; an analysis step of separating and analyzing the pyrolysates using a two-dimensional gas chromatograph mass spectrometer; a table creation step of creating a table by performing assignment of peaks appearing in each two-dimensional chromatogram obtained after the analysis step and alignment between the two-dimensional chromatograms; and a classification step of classifying resins by performing multivariate analysis based on the created table.

In the resin classification method (1), a resin classification method (2) in which in the purification step, a temperature range in which the heat treatment is performed is 100° C. to 300° C. is preferred.

In the resin classification method (1) or (2), a resin classification method (3) in which in the decomposition step, a temperature range in which the pyrolysis treatment is performed is 400° C. to 700° C. is preferred.

In any one of the resin classification methods (1) to (3), a resin classification method (4) in which in the two-dimensional gas chromatograph mass spectrometer, a first column to be used is a non-polar column, and a second column is a medium-polar column or a high-polar column is preferred.

In any one of the resin classification methods (1) to (4), a resin classification method (5) in which the resins are hydrocarbon resins is preferred.

In the analysis by conventional pyrolysis gas chromatography, only some peaks among many generated pyrolysates are assigned and identified as resin monomers, and only rough classification has been performed. This is because a lot of labor is required for assignment of all peaks, and peaks overlap in one-dimensional chromatography, and sufficient analysis cannot be performed.

On the other hand, in the method for classifying resins according to the present invention, it is possible to successfully separate and assign many peaks by using a two-dimensional gas chromatograph mass spectrometer, and it is possible to classify resins in more detail by statistically analyzing the created peak list. Furthermore, the method for classifying resins according to the present invention includes a purification step of removing volatile components by subjecting resins to be classified to a heat treatment at a temperature lower than each decomposition temperature before performing a decomposition step of subjecting the resins to be classified to a pyrolysis treatment at a temperature higher than each decomposition temperature to obtain pyrolysates of the resins. As a result, classification and analysis of the resin main skeleton can be performed without being affected by impurities and an anti-aging agent in the resin, and the resins can be classified more accurately.

### BRIEF DESCRIPTION OF THE DRAWINGS

FIG. 1 is a three-dimensional plot display result of principal component analysis of resins A to J.

### DETAILED DESCRIPTION OF THE PREFERRED EMBODIMENTS

The present invention is a method for classifying at least two or more resins, the method including: a purification step of removing volatile components by subjecting resins to be classified to a heat treatment at a temperature lower than each decomposition temperature; a decomposition step of subjecting the resins to be classified to a pyrolysis treatment at a temperature higher than each decomposition temperature to obtain pyrolysates of the resins; an analysis step of separating and analyzing the pyrolysates using a two-dimensional gas chromatograph mass spectrometer; a table creation step of creating a table by performing assignment of peaks appearing in each two-dimensional chromatogram obtained after the analysis step and alignment between the two-dimensional chromatograms; and a classification step of classifying the resins by performing multivariate analysis based on the created table. Each of the steps will be described below.

The present invention is a method for classifying at least two or more resins. The resins to be classified in the present invention are preferably hydrocarbon resins represented by petroleum resins. Hydrocarbon resins are oligomers or polymers polymerized from olefin monomers (mainly petroleum fractions). For example, when a hydrocarbon resin is blended in a rubber composition which is a raw material of a vulcanized rubber for tires, a line effect such as WETμ can be expected. The resins to be classified are at least two or more resins, and the upper limit of the number of resins is not particularly limited. However, the upper limit of the number of resins to be classified is preferably about 10 or less in order to avoid excessive complexity when assignment of peaks appearing in a two-dimensional chromatogram and alignment between two-dimensional chromatograms are performed in a table creation step to be described later.

### <Purification Step>

In the purification step, volatile components are removed by subjecting resins to be classified to a heat treatment at a temperature lower than each decomposition temperature. In the present invention, the resins are preferably hydrocarbon resins, and in the purification step, heat treatment is preferably performed at a temperature lower than the decomposition temperature of the hydrocarbon resins, specifically, at 100° C. to 300° C. By performing the purification step, volatile components such as low molecular weight compounds mixed in the resins can be removed in the synthesis or separation step of the resins to be classified, and the final classification accuracy of the resins can be improved.

### <Decomposition Step>

In the decomposition step, the resins to be classified are subjected to a pyrolysis treatment at a temperature higher than each decomposition temperature to obtain pyrolysates of the resins. In the present invention, the resins are preferably hydrocarbon resins, and in the decomposition step, the temperature range in which the pyrolysis treatment is performed is preferably 400° C. to 700° C.

<Analysis Step>In the analysis step, the pyrolysates of the resins to be classified are separated and analyzed using a two-dimensional gas chromatograph mass spectrometer. The two-dimensional gas chromatograph can perform temperature programmed analysis by individually controlling the temperature of an independent first column and an independent second column. In the present invention, the first column to be used is preferably a non-polar column, and the second column is preferably a medium-polar column or a high-polar column. The first column and the second column are preferably connected via a modulator. The modulator cryotraps volatile components separated in the first column in a column using cold gas cooled with liquid nitrogen, concentrates peak bands for a certain period of time (about 5 to 10 seconds), and sends the peak bands to the second column.

### <Table Creation Step>

In the table creation step, a table is created by performing assignment of peaks appearing in each two-dimensional chromatogram obtained after the analysis step and alignment between the two-dimensional chromatograms. The table can be created, for example, by taking samples in a vertical arrangement (row) and peaks in a horizontal arrangement (column) and making respective intensities into a matrix.

### <Classification Step>

In the classification step, the resins are classified by performing multivariate analysis based on the created table. Examples of a method for performing multivariate analysis include principal component analysis known to those skilled in the art. The resins to be classified can be classified, for example, by displaying a three-dimensional plot of the result of the principal component analysis.

In the method for classifying resins according to the present invention, it is possible to successfully separate and assign many peaks by using a two-dimensional gas chromatograph mass spectrometer, and it is possible to classify resins in more detail by statistically analyzing the created peak list. Therefore, the present invention is useful as a method for classifying resins, particularly hydrocarbon resins, whose physical properties are greatly different depending on a difference in the type and amount of monomers.

### EXAMPLES

Hereinbelow, the present invention will more specifically be described with reference to examples.

As resins to be classified, ten types of resins including resins A to J were prepared. Resins A to J are all hydrocarbon resins. First, volatile components were removed by a heat treatment at a temperature lower than the decomposition temperature of each of resins A to J (100° C. to 300° C.) (purification step). Next, a pyrolysis treatment was performed at a temperature higher than the decomposition temperature of each of resins A to J (400° C. to 700° C.) to obtain pyrolysates of the resins (decomposition step). Next, the pyrolysates were separated and analyzed using a two-dimensional gas chromatograph mass spectrometer (analysis step). As a two-dimensional gas chromatograph mass spectrometer, “Pegasus4D” manufactured by LECO was used. Measurement conditions are as follows.
(Acquisition Rate) 150 spectra/s
(Acquisition Delay) 1 minute
(Mass Range) 35 to 600 u
(Transfer Line Temp.) 270° C.
(Ion Source Temperature) 250° C.
(First Column (Column 1)) (non-polar column, 30.5 m×0.25 mm, 0.25 μm
(Second Column (Column 2)) (middle-polar column or high-polar column, 1.8 m×0.18 mm, 0.18 μm
(Column 1 Oven) 40° C. 5 min→5° C./min→250° C. 23 min
(Column 2 Oven) 45° C. 5 min→5° C./min→255° C. 23 min
(Modulation Period) 12 s
(Modulator Offset) +15° C.
(Split Ratio) 70
(Injection) Curie Point pyrolyzer, 590° C.
(Carrier Gas) He, 92.3 mL/min (constant flow)

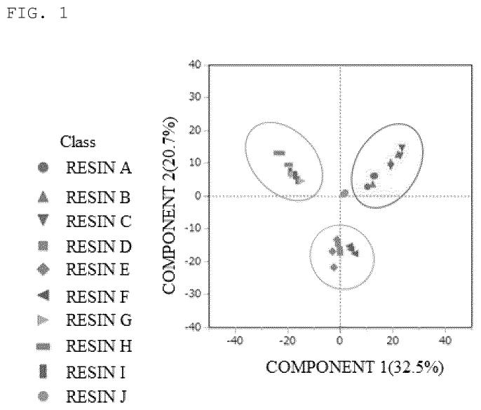

Next, assignment of peaks appearing in each two-dimensional chromatogram obtained after the analysis step and alignment between the two-dimensional chromatograms were performed to form a matrix, thereby creating a table (table creation step). Finally, the resins were classified by performing principal component analysis (multivariate analysis) based on the created table (classification step). FIG. 1 shows three-dimensional plot display results of principal component analysis of resins A to J.

From the results of FIG. 1, it can be seen that resins A to J can be largely classified into three categories of resins A to C, resins D to F, and resins G to I, and resin J is unique.

What is claimed is:

1. A method for classifying at least two or more resins, the method comprising: a purification step of removing volatile components by subjecting resins to be classified to a heat treatment at a temperature lower than each decomposition temperature; a decomposition step of subjecting the resins to be classified to a pyrolysis treatment at a temperature higher than each decomposition temperature to obtain pyrolysates of the resins; an analysis step of separating and analyzing the pyrolysates using a two-dimensional gas chromatograph mass spectrometer; a table creation step of creating a table by performing assignment of peaks appearing in each two-dimensional chromatogram obtained after the analysis step and alignment between the two-dimensional chromatograms; and a classification step of classifying resins by performing multivariate analysis based on the created table.

2. The method for classifying resins according to claim 1, wherein in the purification step, a temperature range in which the heat treatment is performed is 100°C to 300°C.

3. The method for classifying resins according to claim 1, wherein in the decomposition step, a temperature range in which the pyrolysis treatment is performed is 400°C to 700°C.

4. The method for classifying resins according to claim 1, wherein in the two-dimensional gas chromatograph mass spectrometer, a first column to be used is a non-polar column, and a second column is a medium-polar column or a high-polar column.

5. The method for classifying resins according to claim 1, wherein the resins are hydrocarbon resins.
